(19) United States
(12) Patent Application Publication
(10) Pub. No.: US 2021/0333273 A1
(43) Pub. Date: Oct. 28, 2021

(54) LABORATORY TEST KIT AND METHOD FOR USING THE LABORATORY TEST KIT

(71) Applicant: BioMedical Research Institute Co., Ltd., Yokohama-shi (JP)

(72) Inventors: Isao MIYAZAKI, Yokohama-shi (JP); Kazumi HAGA, Tokyo (JP); Kei HAGA, Tokyo (JP)

(21) Appl. No.: 16/318,998

(22) PCT Filed: Oct. 17, 2018
(86) PCT No.: PCT/JP2018/038639

§ 371 Date: Jan. 18, 2019

(30) Foreign Application Priority Data
Mar. 30, 2018 (JP) 2018-070192

(51) Int. Cl. G01N 33/543; G01N 33/569

(52) U.S. Cl. CPC G01N 33/54388; G01N 2333/185; G01N 33/56983

(57) ABSTRACT

A laboratory test kit includes a development part 12 for developing thereon a test sample containing a virus, reagent portion 13 and a recognition portion 17. On the recognition portion 17, first and second specific antibodies both derived from a mouse are to be immobilized for specifically recognizing first and second antigen-determining sites respectively both possessed by the virus. The reagent portion 13 includes first and second labeled antibodies both derived from a mouse, which specifically recognize the first and second antigen-determining sites respectively both possessed by the virus and are labeled with a labeling substance. In each of the first and second labeled antibodies, at least one of an antigen site which is recognized by the HAMA or a sugar chain binding site recognized by an antibody which recognizes a sugar chain is defected or modified.

### TECHNICAL FIELD

The present invention relates to a laboratory test kit and a method for using the laboratory test kit which are capable of diagnosing Dengue caused by the dengue virus.

### BACKGROUND ART

Dengue caused by the dengue virus is one type of infectious diseases caused by viruses. Dengue is classified into dengue fever (DF), dengue hemorrhagic fever and dengue shock syndrome (DHF/DSS). The number of patients with the dengue virus infectious disease and affected areas have been steadily increasing in recent years due to global warming, expanded movement range and shortened movement time of humans and goods, and the like.

Similar to Japanese encephalitis virus, West Nile virus, yellow fever virus, and the like, the dengue virus belongs to the genus Flavivirus in the family Flaviviridae. And, it is known that infection thereof is mediated by Aedes aegypti or Aedes albopictus of the genus Aedes and is expanded by a human (infected person), a mosquito and/or a human.

In addition, according to estimation made by WHO (World Health Organization), among about 2.5 billion people residing in the tropics and subtropics, 50 million people are infected with the dengue virus each year, out of which about 8 million people including 500,000 DHF/DSS patients are hospitalized and approximately 30,000 people, who are mainly children, die every year. Even in Japan, as the number of overseas travelers increases, the number of infected people is increasing year after year as an imported infectious disease. However, therapeutic drugs and vaccines against the dengue virus infectious disease have not yet been developed.

By paying attention to the worldwide number of infected people and expansion of infected areas, WHO has adopted the dengue virus infectious disease as one of Neglected Tropical Diseases. WHO also has been making efforts to take prophylactic or preventive measures against spread of the infection along with development of therapeutic drugs and vaccines. Further, WHO also has been requesting each country to report information such as trends of onset patients and a type (serotype) of the virus.

Therefore, for the purpose of knowing trends of onset patients at an early stage and for the purpose of making a decision for a therapeutic policy at an early stage, it is required to develop a test method which makes it possible to know whether dengue virus infection occurs or not at early days after onset.

Here, in recent years, as such a test method which is capable of diagnosing Dengue caused by the dengue virus in a convenient, rapid and inexpensive manner, a test method using an immunochromatography system in which an antigen-antibody reaction is utilized has been widely used (see, for example, PTD 1).

Such a test method using the immunochromatography system is applied even with respect to dengue virus infection. However, in this case, it is hard to say that Dengue can be diagnosed at an early days (day 0 to 3) after onset with excellent detection sensitivity, especially secondly infection.

In addition, it cannot also be said that viruses other than the dengue virus can be detected at an early stage with excellent detection sensitivity.

### CITATION LIST

### Patent Document

[PTD 1] PCT Japanese Translation Patent Publication No. 2003-511697

### SUMMARY OF INVENTION

### Technical Problem

An object of the present invention is to provide a laboratory test kit and a test method for using the laboratory test kit which are capable of diagnosing infection caused by a virus at early days after onset with excellent detection sensitivity.

### Means for Solving the Problem

The object is achieved by the present invention described in the following (1) to (9).

(1) A laboratory test kit used for diagnosing infection caused by a virus, comprising a development part for developing thereon a test sample containing the virus, a recognition portion provided in the middle of the development part, and a reagent portion disposed in contact with an edge portion of an upstream side of the development part when a developing direction in which the test sample is developed on the development part is taken as a reference,

the recognition portion on which a first specific antibody and a second specific antibody both derived from a mouse are to be immobilized for specifically recognizing a first antigen-determining site and a second antigen-determining site both possessed by the virus,

the reagent portion includes a first labeled antibody and a second labeled antibody both derived from a mouse, which have a function of specifically recognizing the first antigen-determining site and the second antigen-determining site both possessed by the virus and are labeled with a labeling substance, and

in each of the first labeled antibody and the second labeled antibody, at least one of an antigen site which is recognized by a human anti-mouse antibody or a sugar chain binding site recognized by an antibody which recognizes a sugar chain is defected or modified.

(2) The laboratory test kit as defined by (1), wherein in each of the first specific antibody and the second specific antibody, at least one of an antigen site which is recognized by the human anti-mouse antibody or a sugar chain binding site recognized by the antibody which recognizes the sugar chain is defected or modified.

(3) The laboratory test kit as defined by (1) or (2), wherein the virus is dengue virus.

(4) The laboratory test kit as defined by (3), wherein each of the first specific antibody and the first labeled antibody specifically recognizes a part of an internal antigen possessed by the dengue virus.

(5) The laboratory test kit as defined by (4), wherein the internal antigen is an E-protein contained in an envelope.

(6) The laboratory test kit as defined by any one of (3) to (5), wherein each of the second specific antibody and the second labeled antibody specifically recognizes a part of an external antigen possessed by the dengue virus.

(7) The laboratory test kit as defined by (6), wherein the external antigen is a NS1 produced by the dengue virus at the outside of the virus.

(8) The laboratory test kit as defined by any one of (1) 1 to (7), further comprises a sample supply portion disposed so as to be in contact with the edge portion of the upstream side of the reagent portion, and a blood-corpuscles filtering portion disposed so as to be laminated on the sample supply portion, wherein the test sample is whole blood,

wherein the laboratory test kit is configured so that by supplying the test sample into the sample supply portion through the blood-corpuscles filtering portion, the blood-corpuscles components contained in the test sample are eliminated.

(9) A method for diagnosing virus infection using the laboratory test kit defined by any one of (1) to (8), comprising:

a first step for supplying the test sample onto the developing layer; and

a second step for confirming whether or not the virus is captured by the recognition portion provided in the middle of the development part when the test sample is developed onto the development part.

### Effect of the Invention

According to the present invention, it is possible to diagnose the infection caused by the virus at early days after onset with excellent detection sensitivity. Especially, it is possible to diagnose Dengue caused by the dengue virus at early days after onset with excellent detection sensitivity.

In this way, to diagnose virus infection at early days after onset can be carried out with excellent accuracy, and a therapeutic policy can be established at early days after onset. Therefore, in a case where such a test for diagnosing virus infection is applied to the diagnosis of dengue virus infection, it is possible to reduce onset of dengue hemorrhagic fever/dengue shock syndrome (DHF/DSS), and furthermore, it is also possible to reduce a fatality rate caused by the DHF/DSS onset.

### BRIEF DESCRIPTION OF DRAWINGS

FIG. 1 illustrates an embodiment of the laboratory test kit of the present invention (in which FIG. 1(a) is a plan view and FIG. 1(b) is a longitudinal sectional view).

FIG. 2 illustrates an embodiment of an immunochromatography test piece included in the laboratory test kit of the present invention (in which FIG. 2(a) is a perspective view and FIG. 2(b) is an exploded perspective view).

### DESCRIPTION OF EMBODIMENTS

Hereinafter, the laboratory test kit and the method for using the laboratory test kit of the present invention will be described in detail based on suitable embodiments shown in the accompanying drawings.

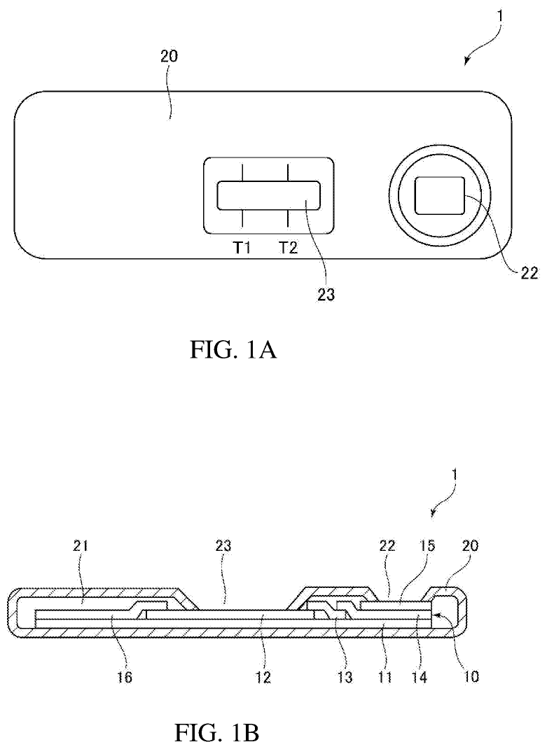

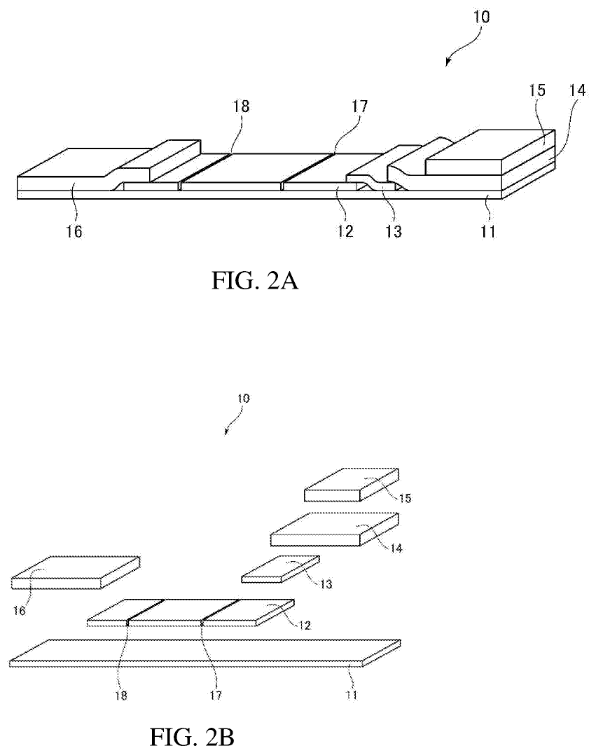

FIG. 1 illustrates an embodiment of the laboratory test kit of the present invention (in which FIG. 1(a) is a plan view and FIG. 1(b) is a longitudinal sectional view), and FIG. 2 illustrates an embodiment of an immunochromatography test piece included in the laboratory test kit of the present invention (in which FIG. 2(a) is a perspective view and FIG. 2(b) is an exploded perspective view). For the sake of convenience of description, in the following description, upper sides of FIG. 1(a) and FIG. 2 are referred to as “upper” and lower sides thereof are referred to as “lower”.

First, the laboratory test kit of the present invention will be described.

The laboratory test kit of the present invention is used for diagnosing virus infection using an immunochromatography system.

Here, the immunochromatography system is generally a method of detecting a virus of an object to be tested in a test sample (specimen) through the following steps. That is, the immunochromatography system is a method in which 1: first, a test sample (specimen) is brought into contact with a labeled antibody that specifically recognizes a virus antigen as an object to be tested; 2: next, the test sample develops along a development part due to a capillary phenomenon, and, during the development, a complex of the object to be tested and the labeled antibody is captured by a specific antibody which is immobilized in the middle of the development part and specifically recognizes the object to be tested, and 3: the virus of the object to be tested can be detected by observing the labeled antibody which is included in the captured complex.

According to such a method, the object to be tested contained in the test sample can be detected (analyzed) by a simple operation such as dropping the test sample such as blood (whole blood) or urine onto a test piece including a development part. Therefore, in recent years, the immunochromatography system has been used as a very useful technique for conveniently and rapidly detecting an object to be tested which is contained in a test sample.

Hereinafter, the laboratory test kit of the present invention which is capable of diagnosing the virus infection using such an immunochromatography system will be described.

In the following, a case of detecting the dengue virus belonging to the family Flaviviridae, which is one type of viruses, using the laboratory test kit of the present invention will be described by way of an example.

The laboratory test kit 1 shown in FIG. 1 has an immunochromatography test piece 10 for diagnosing the dengue virus infection and a housing case (casing) 20 for accommodating the immunochromatography test piece 10.

As shown in FIG. 2, the immunochromatography test piece 10 (hereinafter simply referred to as “test piece”) has a support substrate 11, on which disposed respectively are a blood cell filtration portion (blood-corpuscles filtering portion) 15, a sample supply portion 14, a reagent portion 13, a development part 12 (with a recognition portion 17, a control portion 18) and an absorption portion 16. The blood cell filtration portion 15 filters blood cell components in a test sample. The sample supply portion 14 supplies the test sample in which the blood cell components have been filtered. The reagent portion 13 includes a labeled antibody that specifically recognizes the dengue virus antigens contained in the test sample. The development part 12 develops the test sample. The recognition portion 17 is provided in the middle of the development part 12 and on which a specific antibody that specifically recognizes the dengue virus antigens contained in the test sample is immobilized. The control portion 18 is provided on the opposite side of the reagent portion 13 of the recognition portion 17 in the middle of the development part 12 and on which an anti-antibody (anti-labeled antibody antibody) that recognizes the labeled antibody is immobilized. And, The absorption portion 16 absorbs the test sample which has been developed along the development part 12.

Here, a development direction in which the test sample is developed along the development part 12 is taken as a reference. In the test piece 10, the sample supply portion 14 is disposed via the reagent portion 13 on an upstream side of the development part 12, and the absorption portion 16 is disposed on a downstream side of the development part 12. In other words, from the upstream side to the downstream side in the development direction on the support substrate 11, the sample supply portion 14, the reagent portion 13, the development part 12, and the absorption portion 16 are disposed in this order such that edge portions thereof come into contact with each other.

The support substrate 11 is in a strip (flat plate) shape and supports various members constituting the test piece 10, that is, the blood cell filtration portion 15, the sample supply portion 14, the reagent portion 13, the development part 12, and the absorption portion 16.

As a constituent material for the support substrate 11, examples thereof can include a resin material such as polystyrene, polycarbonate, polypropylene, polyester, cellulose acetate, and polyethylene terephthalate, or the like. Among these, one type can be used or two or more types can be used in combination.

A thickness of the support substrate 11 is not particularly limited, and is, for example, preferably about 0.05 to 0.6 mm, and more preferably about 0.1 to 0.5 mm.

The blood cell filtration portion 15 is in a strip shape in view of an overall shape thereof, and is disposed so as to be stacked on the sample supply portion 14. The blood cell filtration portion 15 filters (separates) blood cell components. Thus, the blood cell filtration portion 15 can supplies the test sample in which the blood cell components have been filtered, that is, plasma components, to the sample supply portion 14.

By causing the test piece 10 to be configured to have the blood cell filtration portion 15 in this way, it is possible to use whole blood or the like containing blood cell components such as red blood cells, white blood cells and platelets as a test sample for detecting the dengue virus. Therefore, it is possible to decrease labor and time taken at the time of separating plasma components from whole blood or the like using centrifugation or the like.

The blood cell filtration portion 15 is usually composed of a filtration membrane such as a glass filter, and a pore diameter thereof is preferably set at about 100 to 1,500 nm, and more preferably set at about 200 to 1,000 nm. This is because a size of dengue virus is about 40 to 70 nm and a size of blood cell components is about 2,000 μm or more. Thus, by setting the pore diameter of the filtration membrane to be within such a range, it is possible to bring the dengue virus to a plasma component side while filtering blood cell components. Therefore, it is possible to decrease a capture rate that the dengue virus is captured in the blood cell filtration portion 15 and to improve a development rate that the dengue virus develops along the development part 12. For these reasons, it is possible to improve the detection sensitivity at the time of diagnosing the dengue virus infection by using the test piece 10.

In a case where, whole blood is not used but plasma components and serum components obtained by previously removing blood cell components from whole blood are used as a test sample, it is possible to omit to provide the blood cell filtration portion 15 on the sample supply portion 14.

The sample supply portion 14 is in a strip shape in view of an overall shape thereof. The sample supply portion 14 is disposed on the support substrate 11 so that an edge portion of the sample supply portion 14 on a downstream side in a longitudinal direction thereof contacts (overlaps) the reagent portion 13. Thus, the sample supply portion 14 supplies the test sample, which is added via the blood cell filtration portion 15, to the reagent portion 13.

The sample supply portion 14 is not particularly limited as long as the portion has liquid permeability. For example, the sample supply portion 14 is formed from a porous material and a fibrous material. In a case where the sample supply portion 14 is formed from the porous material or the fibrous material, porosity of the sample supply portion 14 may have following features. That is, the porosity of the sample supply portion 14 may have uni-directionality in which a communication direction of pores or a stretching direction of fibers is parallel with respect to a longitudinal direction of the sample supply portion 14. Further, the porosity of the sample supply portion 14 may have multi-directionality in which a communication direction of pores or a stretching direction of fibers is random with respect to a longitudinal direction of the sample supply portion 14.

As a constituent material for such a porous material and fibrous material, examples thereof can include resin materials such as glass wool, polypropylene, polyethylene, polyvinylidene fluoride, ethylene vinyl acetate, acrylonitrile and polytetrafluoroethylene in addition to cellulose materials such as paper and nitrocellulose. Among these, one type can be used or two or more types can be used in combination.

The reagent portion 13 is in a strip shape in view of an overall shape thereof. The reagent portion 13 is disposed on the support substrate 11 so that one edge portion of the reagent portion 13 on an upstream side in a longitudinal direction thereof contacts (overlaps) the sample supply portion 14, and the other edge portion thereof on a downstream side contacts the development part 12. Thus, the reagent portion 13 supplies the test sample, which is supplied via the sample supply portion 14, to the development part 12.

Furthermore, the reagent portion 13 has, as a reagent, a labeled antibody which has a function of specifically recognizing the dengue virus antigens and is labeled with a labeling substance. In the reagent portion 13, the labeled antibody is held (retained) on a porous carrier in a state of being releasable by passage (permeation) of a liquid. That is, the reagent portion 13 contains, as a reagent, the labeled antibody which has a function of specifically recognizing the dengue virus antigens and is labeled with a labeling substance.

Therefore, in a case where the test sample permeates (passes) through the reagent portion 13 and is supplied to the development part 12, the test sample is in a state where the labeled antibody (reagent) is added therein.

In addition, in a case where the dengue virus antigen is contained in the test sample, that is, in a case (positive case) where a subject is infected with the dengue virus, since the labeled antibody specifically recognizes the dengue virus antigen, a complex composed of the labeled antibody and the dengue virus antigen is formed. Thus, the test sample is supplied to the development part 12 in a state where such a complex is formed.

The porous carrier included in the reagent portion 13 is not particularly limited as long as the carrier is liquid permeable. For example, the carrier is formed from a porous material and a fibrous material as well as the sample supply portion 14.

In addition, the labeled antibody is an antibody obtained by labeling an antibody (secondary antibody), which specifically recognizes the dengue virus antigen, with a labeling substance. However, the antibody (secondary antibody) is not particularly limited. For example, any of IgG, IgA, IgM, IgE and IgD may be used, with IgG being preferably used from the viewpoint of ease of handling.

Furthermore, in the present invention, as the antibody (secondary antibody), a mouse-derived antibody (mouse antibody) is used, which may be either a monoclonal antibody or a polyclonal antibody, with a monoclonal antibody being preferable.

In addition, as the above-mentioned antibody (secondary antibody), an antibody that specifically recognizes the dengue virus antigens is used. In the present embodiment, the antibody (secondary antibody) contains a first antibody (first secondary antibody) and a second antibody (second secondary antibody). The first antibody specifically recognizes, as an antigen-determining site (first antigen-determining site; epitope), a part of an external antigen (nonstructural protein) such as NS1, NS2a, NS2B, NS3, NS4a, NS4B and NS5 which are produced by the dengue virus and are released to the outside of the virus. And, the second antibody specifically recognizes, as an antigen-determining site (second antigen-determining site), a part of an internal antigen (structural protein) such as E protein, M protein and C protein which are included in the envelope.

Here, for example, NS1 which is one type of external antigens is a protein contained in the blood for a period (test period) from about 2 to 3 days to 10 days after the onset of Dengue caused by the dengue virus. Furthermore, the E protein which is one type of internal antigens is a protein contained in the blood for a period from about 0 days to 5 to 7 days after the onset of Dengue caused by the dengue virus. The NS1 and the E protein are different with respect to a period contained in the blood.

Therefore, it is possible to choose, as the first antibody (first secondary antibody), an antibody that recognizes a part of NS1 which is one type of external antigens as an antigen-determining site, while also choosing, as the second antibody (second secondary antibody), an antibody that recognizes a part of the E protein which is one type of internal antigens as an antigen-determining site. For this reason, the labeled antibody that includes both of these first secondary antibody and second secondary antibody as secondary antibodies can bind to an antigen-determining site included in an antigen contained in the test sample for a period from about 0 days to 10 days which is set as a window size (optimally testable period), so that the antigen derived from the dengue virus is recognized. In this way, two types of the first secondary antibody and the second secondary antibody having different window sizes can be used as the secondary antibody included in the labeled antibody. That is, two types of the first labeled antibody and the second labeled antibody which have functions of specifically recognizing a first antigen-determining site and a second antigen-determining site, respectively, possessed by the dengue virus and are labeled with a labeling substance can be used as the labeled antibody. This makes it possible to increase a window size period as compared with a case where any one of the two types is used as the secondary antibody. Therefore, the detection of the dengue virus by the laboratory test kit 1 can be carried out with high sensitivity over a long period from early days after onset caused by the dengue virus.

Furthermore, as described above, a mouse antibody can be used as the antibody (secondary antibody) included in the labeled antibody. In this case, among the oriental people, for about 10% to 15% of Japanese people, and about 20% to 25% of Thai people, it is said that a human anti-mouse antibody (HAMA) which is one type of heterophilic antibodies is contained in the blood and the HAMA activity is possessed. Therefore, in a case where the HAMA is contained in a test sample (blood), the HAMA recognizes the secondary antibody, and aggregation occurs between the labeled antibody including the secondary antibody and the HAMA. As a result, an erroneous diagnosis may occur due to a false positivity which is determined to be positive despite the fact that infection caused by the dengue virus is not confirmed.

In addition, in a case where a mouse antibody is used as the antibody (secondary antibody) included in the labeled antibody, a sugar chain is unavoidably bound to the constant region of the mouse antibody. Therefore, in a case where a human antibody that recognizes the sugar chain is contained in a test sample (blood), the human antibody recognizes the secondary antibody, and aggregation occurs between the labeled antibody including the secondary antibody and the human antibody. As a result, even in a case where the human antibody is contained, an erroneous diagnosis may occur due to a false positivity which is determined to be positive despite the fact that infection caused by the dengue virus is not confirmed.

On the contrary, according to the present invention, the reagent portion 13 includes, as the antibody (secondary antibody) included in the labeled antibody, an antibody in which at least one of an antigenic site (antigen site) recognized by the HAMA or a binding site of a sugar chain recognized by the human antibody which are contained in the constant region of the antibody (secondary antibody) is defected or modified. That is, in each of the first labeled antibody and the second labeled antibody which are labeled with a labeling substance, at least one of the antigenic site recognized by the HAMA or the binding site of a sugar chain recognized by the human antibody is defected or modified.

Therefore, in a case where the antigenic site recognized by the HAMA is defected in each of the first labeled antibody and the second labeled antibody which are labeled with a labeling substance, even though the HAMA is contained in a test sample (blood), aggregation between the labeled antibody including the secondary antibodies and the HAMA is properly prevented from occurring. As a result, a false positivity which is determined to be positive when not confirming infection caused by the dengue virus can be reliably prevented from occurring.

In addition, in a case where the sugar chain binding site is defected or modified in each of the first labeled antibody and the second labeled antibody which are labeled with a labeling substance, even though a human antibody that recognizes a sugar chain is contained in a test sample (blood), aggregation between the labeled antibody including the secondary antibodies and the human antibody that recognizes a sugar chain is properly prevented from occurring. As a result, a false positivity which is determined to be positive when not confirming infection caused by the dengue virus can be reliably prevented from occurring.

As described above, in the present invention, at least one of the antigenic site recognized by the HAMA or the sugar chain binding site recognized by the antibody that recognizes a sugar chain is defected or modified in each of the first labeled antibody and the second labeled antibody which are labeled with a labeling substance. Accordingly, a false positivity which is determined to be positive when not confirming infection caused by the dengue virus can be reliably prevented from occurring. As a result, it is possible to improve the detection sensitivity of the laboratory test kit 1.

In the antibody (secondary antibody) included in the labeled antibody, at least one of the antigenic site recognized by the HAMA or the sugar chain binding site recognized by the human antibody which are contained in the constant region of the antibody (secondary antibody) is defected or modified, and it is preferable that both of the antigenic site and the sugar chain binding site are defected or modified. Accordingly, it is possible to obtain both an effect obtained in a case where the antigenic site recognized by the HAMA is defected or modified and an effect obtained in a case where the sugar chain binding site recognized by the human antibody is defected or modified.

In addition, according to the present embodiment, in the constant region (more specifically, CH2 region) of the antibody (secondary antibody) included in the labeled antibody, at least one of the antigenic site recognized by the HAMA or the sugar chain binding site recognized by the human antibody is defected or modified. Among these, the defected or modified site of the antigenic site recognized by the HAMA may be provided in the variable site (V region) other than the constant region (C region) of the antibody (secondary antibody) included in the labeled antibody. However, it is preferable for the defected or modified site to be provided in the constant region (C region) as in the present embodiment. Accordingly, it is possible to properly suppress or prevent decrease in the recognition ability of the first antibody (first secondary antibody) to recognize, as an antigen-determining site, a part of NS1 which is one type of external antigens. In addition, it is possible to properly suppress or prevent decrease in the recognition ability of the second antibody (second secondary antibody) to recognize, as an antigen-determining site, a part of the E protein which is one type of internal antigens.

Furthermore, in a case where both the antigenic site recognized by the HAMA and the sugar chain binding site recognized by the human antibody are defected or modified in the constant region of the antibody (secondary antibody) included in the labeled antibody, it is preferable that these sites of the defect or modification are adjacent or partially overlapped with each other. Accordingly, it is possible to simplify a step carried out in a case of obtaining the first antibody (first secondary antibody) and the second antibody (second secondary antibody) as mouse-derived antibodies (mouse antibodies). Furthermore, it is possible to properly suppress or prevent decrease in the recognition ability of the first antibody (first secondary antibody) to recognize, as an antigen-determining site, a part of NS1 which is one type of external antigens. In addition, it is possible to properly suppress or prevent decrease in the recognition ability of the second antibody (second secondary antibody) to recognize, as an antigen-determining site, a part of the E protein which is one type of internal antigens.

As a method for labeling an antibody, there is no particular limitation. Examples thereof can include a colloidal gold label, a colored latex particle label, a radioactive label, a fluorescent dye label, an enzyme label, and the like. Among these, one type can be used, or two or more types can be used in combination, and the colloidal gold label is preferably used. With the colloidal gold label, it becomes possible to visually recognize a complex composed of the labeled antibody and the dengue virus antigen with excellent sensitivity.

In a case where the colloidal gold label is used as a method for labeling an antibody, labeling of an antibody with colloidal gold is carried out, for example, by mixing the antibody and the colloidal gold in a buffer solution. According to such a method, the antibody is physically adsorbed to the colloidal gold so that the antibody is labeled.

In the labeled antibody contained in the reagent portion 13, a mixing ratio (molar ratio) of the first labeled antibody to the second labeled antibody is preferably about 1:0.1 to 1:20, and more preferably about 1:0.5 to 1:10. Accordingly, the first labeled antibody and the second labeled antibody specifically recognize a first antigen-determining site and a second antigen-determining site which are possessed by the dengue virus, respectively. Therefore, a complex of the first labeled antibody and the dengue virus antigen, and a complex of the second labeled antibody and the dengue virus antigen can be reliably formed.

Furthermore, as a method of causing the labeled antibody (reagent) to be held on a porous carrier, that is, a method of forming the reagent portion 13, examples thereof can include the following method. That is, an aqueous solution containing the labeled antibody is prepared, the prepared aqueous solution is impregnated in the porous carrier, and then drying is carried out. According to such a method, it is possible to cause the labeled antibody to be uniformly held on the porous carrier.

The development part 12 is in a strip shape in view of an overall shape thereof, and is disposed on the support substrate 11 such that one edge portion of the development part 12 on an upstream side in a longitudinal direction thereof contacts (overlaps) the reagent portion 13, and the other edge portion thereof on a downstream side contacts the absorption portion 16. The development part 12 develops the test sample, which is supplied from the reagent portion 13 and contains the labeled antibody (reagent), from the edge portion on the upstream side to the other edge portion on the downstream side due to a capillary phenomenon.

The development part 12 is usually composed of a porous carrier so that the test sample can be developed by a capillary phenomenon. Specifically, for example, the development part 12 is composed of a membrane formed of cellulose or a cellulose derivative such as cellulose acetate and nitrocellulose, a glass filter, a filter paper, or the like. Among these, it is preferable that the development part 12 is composed of the nitrocellulose membrane. By causing the development part 12 to be formed of the porous carrier composed of the nitrocellulose membrane, it is possible to easily carry out immobilization of a specific antibody in the recognition portion 17 as described later and immobilization of an anti-antibody in the control portion 18.

In addition, a pore diameter of the development part 12 is preferably 1 μm or more, more preferably 5 μm or more, and even more preferably 8 μm to 12 μm. Accordingly, not only liquid components but also the dengue virus, the labeled antibody and the complex of these contained in the test sample can be reliably permeated through the pores. Therefore, it becomes possible to diagnose the dengue virus infection with higher sensitivity.

In addition, for the development part 12 having the above-described configuration, in the middle thereof, there are provided the recognition portion 17 and the control portion 18 which is disposed on a downstream side of the recognition portion 17.

The recognition portion 17 is a portion on which a specific antibody (primary antibody) that specifically recognizes the dengue virus antigens is immobilized in the middle of the development part 12 on an upstream side of the control portion 18.

In the present embodiment, the recognition portion 17 is formed in a band shape on the upstream side of the control portion 18 in the development part 12 so as to be orthogonal to the longitudinal direction of the development part 12 as shown in FIG. 2.

In a case where a test sample containing the dengue virus, that is, a test sample (positive test sample) derived from a subject infected with the dengue virus is supplied from the reagent portion 13 to the development part 12 which is provided with the recognition portion 17 having such a configuration, the dengue virus infection can be diagnosed in recognition portion 17 as follows.

That is, in a case where the dengue virus is contained in the test sample, the specific antibody (primary antibody) immobilized on the recognition portion 17 recognizes the dengue virus. Therefore, the dengue virus is captured in the recognition portion 17.

Furthermore, as stated in the description of the reagent portion 13, in the test sample which has permeated through the reagent portion 13 and has been supplied to the development part 12, the complex composed of the dengue virus antigen and the labeled antibody have been formed. For this reason, when the dengue virus antigen is recognized (captured) in the recognition portion 17, the labeled antibody is also captured in a state of forming the complex together with the dengue virus antigen.

Therefore, a reaction between the immobilized specific antibody and the complex is confirmed by observing whether or not the labeled antibody is captured in the recognition portion 17.

Therefore, it can be said that the test sample contains the dengue virus (the subject is infected with the dengue virus) by observing that the labeled antibody is captured in the recognition portion 17.

As described above, the infection caused by the dengue virus can be diagnosed.

As the specific antibody (primary antibody) immobilized on the recognition portion 17, as well as the secondary antibody included in the labeled antibody, there is no particular limitation as long as the antibody specifically recognizes the dengue virus antigen in the recognition portion 17. For example, any of IgG, IgA, IgM, IgE and IgD may be used, and IgG is preferably used from the viewpoint of ease of handling.

Furthermore, in the present invention, as the specific antibody (primary antibody), a mouse-derived antibody (mouse antibody) is used, which may be either a monoclonal antibody or a polyclonal antibody, with a monoclonal antibody being preferable.

In addition, as such a specific antibody (primary antibody), similar to the above-mentioned secondary antibody, an antibody that specifically recognizes the dengue virus antigens is used. In the present embodiment, the specific antibody (primary antibody) includes a first specific antibody (first primary antibody) and a second specific antibody (second primary antibody). The first specific antibody specifically recognizes, as an antigen-determining site (first antigen-determining site), a part of an external antigen (nonstructural protein) such as NS1, NS2a, NS2B, NS3, NS4a, NS4B, and NS5. And, the second specific antibody specifically recognizes, as an antigen-determining site (second antigen-determining site), a part of an internal antigen (structural protein) such as E protein, M protein and C protein which are included in the envelope.

Here, as described above, for example, NS1 which is one type of external antigens is a protein contained in the blood for a period from about 2 to 3 days to 10 days after the onset of Dengue caused by the virus. Furthermore, the E protein which is one type of internal antigens is a protein contained in the blood for a period from about 0 days to 5 to 7 days after the onset of Dengue caused by the virus. The NS1 and the E protein are different with respect to a period contained in the blood.

Therefore, it is possible to choose, as the first specific antibody (first primary antibody), an antibody that recognizes a part of NS1 which is one type of external antigens as an antigen-determining site, while also choosing, as the second specific antibody (second primary antibody), an antibody that recognizes a part of the E protein which is one type of internal antigens as an antigen-determining site. For this reason, the specific antibody that includes both of these first specific antibody and second specific antibody can bind to an antigen-determining site included in an antigen contained in the test sample for a period from about 0 days to 10 days which is set as a window size (optimally testable period), so that an antigen derived from the dengue virus is recognized. In this way, two types of the first specific antibody and the second specific antibody having different window sizes can be used as the specific antibody immobilized on the recognition portion 17. That is, two types of the first specific antibody and the second specific antibody which have functions of specifically recognizing a first antigen-determining site and a second antigen-determining site, respectively, possessed by the dengue virus can be used as the specific antibody. This makes it possible to increase a window size period as compared with a case where any one of the two types is used as the specific antibody. Therefore, the detection of the dengue virus by the laboratory test kit 1 can be carried out with high sensitivity over a long period from early days after onset caused by the dengue virus.

Furthermore, as described above, a mouse antibody can be used as the specific antibody (primary antibody) immobilized on the recognition portion 17. Therefore, in a case where the HAMA is contained in the test sample (blood), the HAMA recognizes the specific antibody, and aggregation occurs between the specific antibody and the HAMA. As a result, an erroneous diagnosis may occur due to a false positivity which is determined to be positive despite the fact that infection caused by the dengue virus is not confirmed.

In addition, in a case where a mouse antibody is used as the specific antibody (primary antibody) immobilized on the recognition portion 17, a sugar chain is bound to the constant region of the mouse antibody as described above. Therefore, in a case where a human antibody that recognizes a sugar chain is contained in a test sample (blood), the human antibody recognizes the specific antibody, and aggregation occurs between the specific antibody and the human antibody. As a result, even in a case where the human antibody is contained, an erroneous diagnosis may occur due to a false positivity which is determined to be positive despite the fact that infection caused by the dengue virus is not confirmed.

On the contrary, according to the present embodiment, the recognition portion 17 preferably includes, as the specific antibody (primary antibody) immobilized thereon, an antibody in which at least one of an antigenic site recognized by the HAMA or a binding site of a sugar chain recognized by the human antibody which are contained in the constant region of the specific antibody is defected or modified. That is, in each of the first specific antibody and the second specific antibody which are immobilized on the recognition portion 17, it is preferable that at least one of the antigenic site recognized by the HAMA or the binding site of a sugar chain recognized by the human antibody is defected or modified.

Therefore, in a case where the antigenic site recognized by the HAMA is defected in each of the first specific antibody and the second specific antibody which are immobilized on the recognition portion 17, even though the HAMA is contained in a test sample (blood), aggregation between the specific antibodies immobilized on the recognition portion 17 and the HAMA is properly prevented from occurring. As a result, a false positivity which is determined to be positive when not confirming infection caused by the dengue virus can be reliably prevented from occurring.

In addition, in a case where the sugar chain binding site recognized by an antibody that recognizes a sugar chain is defected in each of the first specific antibody and the second specific antibody which are immobilized on the recognition portion 17, even though a human antibody that recognizes a sugar chain is contained in a test sample (blood), aggregation between the specific antibodies and the human antibody that recognizes a sugar chain is properly prevented from occurring. As a result, a false positivity which is determined to be positive when not confirming infection caused by the dengue virus can be reliably prevented from occurring.

As described above, at least one of the antigenic site recognized by the HAMA or the sugar chain binding site is defected or modified in each of the first specific antibody and the second specific antibody which are immobilized on the recognition portion 17. Accordingly, a false positivity which is determined to be positive when not confirming infection caused by the dengue virus can be reliably prevented from occurring. As a result, it is possible to improve the detection sensitivity of the laboratory test kit 1.

In the specific antibody (primary antibody) immobilized on the recognition portion 17, at least one of the antigenic site recognized by the HAMA or the sugar chain binding site recognized by the human antibody which are contained in the constant region of the specific antibody (primary antibody) is preferably defected or modified, and it is more preferable that both of the antigenic site and the sugar chain binding site are defected or modified. Accordingly, it is possible to obtain both an effect obtained in a case where the antigenic site recognized by the HAMA is defected or modified and an effect obtained in a case where the sugar chain binding site recognized by the human antibody is defected or modified.

In addition, according to the present embodiment, in the constant region (more specifically, CH2 region) of the specific antibody (primary antibody) immobilized on the recognition portion 17, at least one of the antigenic site recognized by the HAMA or the sugar chain binding site recognized by the human antibody is defected or modified. Among these, the defected or modified site of the antigenic site recognized by the HAMA may be provided in the variable site (V region) other than the constant region (C region) of the specific antibody (primary antibody) immobilized on the recognition portion 17. However, it is preferable for the defected or modified site to be provided in the constant region (C region) as in the present embodiment. Accordingly, it is possible to properly suppress or prevent decrease in the recognition ability of the first specific antibody (first primary antibody) to recognize, as an antigen-determining site, a part of NS1 which is one type of external antigens. In addition, it is possible to properly suppress or prevent decrease in the recognition ability of the second specific antibody (second primary antibody) to recognize, as an antigen-determining site, a part of the E protein which is one type of internal antigens.

Furthermore, in a case where both the antigenic site recognized by the HAMA and the sugar chain binding site recognized by the human antibody are defected or modified in the constant region of the specific antibody (primary antibody) immobilized on the recognition portion 17, it is preferable that these sites of the defect or modification are adjacent or partially overlapped with each other. Accordingly, it is possible to simplify a step carried out in a case of obtaining the first specific antibody (first primary antibody) and the second specific antibody (second primary antibody) as mouse-derived antibodies (mouse antibodies). Furthermore, it is possible to properly suppress or prevent decrease in the recognition ability of the first specific antibody (first primary antibody) to recognize, as an antigen-determining site, a part of NS1 which is one type of external antigens. In addition, it is possible to properly suppress or prevent decrease in the recognition ability of the second specific antibody (second primary antibody) to recognize, as an antigen-determining site, a part of the E protein which is one type of internal antigens.

In the specific antibody immobilized on the recognition portion 17, a mixing ratio (molar ratio) of the first specific antibody to the second specific antibody is preferably about 1:0.1 to 1:20, and more preferably about 1:0.5 to 1:10. Accordingly, the first specific antibody and the second specific antibody specifically recognize a first antigen-determining site and a second antigen-determining site which are possessed by the dengue virus, respectively. Therefore, a complex of the first specific antibody and the dengue virus antigen, and a complex of the second specific antibody and the dengue virus antigen can be reliably formed in the recognition portion 17.

In addition, in a case where the development part 12 is the porous carrier composed of the nitrocellulose membrane as described above, immobilization of the specific antibody in the recognition portion 17 is carried out by dropping an aqueous solution containing the specific antibody on a region where the recognition portion 17 is to be formed on the development part 12, and then carrying out drying and washing. By causing the development part 12 to be composed of the nitrocellulose in this way, chemical bonding is formed between the development part 12 and the specific antibody without carrying out a chemical treatment to the development part 12 or the specific antibody in advance. Therefore, by a simple process of bringing the aqueous solution containing the specific antibody into contact with the development part 12, the specific antibody is reliably immobilized on the development part 12, so that the recognition portion 17 can be formed.

A temperature at which the aqueous solution is dried is not particularly limited. For example, the temperature is preferably about 40° C. to 70° C., and more preferably about 50° C. to 65° C. Accordingly, it is possible to reliably immobilize the specific antibody on the development part 12 while suppressing or preventing inactivation of the antigen recognition ability included in the specific antibody.

In addition, in a case where the development part 12 is a porous carrier composed of the filter paper, the immobilization can be carried out by forming a chemical bond using CNBr, carbonyl diimidazole, tresyl chloride, or the like between the development part 12 and the specific antibody.

As described above, the infection caused by the dengue virus can be diagnosed using the laboratory test kit 1.

Thus, based on a test result obtained by the laboratory test kit 1, early diagnosis of the dengue virus infection can be carried out with excellent accuracy, and a therapeutic policy can be established at an early stage. Therefore, it is possible to reduce onset of the dengue hemorrhagic fever/dengue shock syndrome (DHF/DSS). Furthermore, it is possible to reduce a fatality rate caused by the DHF/DSS onset.

In the control portion 18, an anti-antibody (anti-labeled antibody antibody) that recognizes the labeled antibody is immobilized on a downstream side of the recognition portion 17 in the middle of the development part 12.

In the present embodiment, the control portion 18 is formed in a band shape so as to be orthogonal to the longitudinal direction of the development part 12.

By observing a reaction between the immobilized anti-antibody and the labeled antibody in the control portion 18 having such a configuration, it is possible to obtain an evidence which shows that the labeled antibody contained in the test sample which is supplied to the development part 12 develops from the upstream side of the development part 12 to the control portion 18 positioned on the downstream side thereof. Therefore, by observing such a reaction in the control portion 18, it is shown that the detection result (determination result) confirmed by the recognition portion 17 is correct.

In addition, as the anti-antibody, there is no particular limitation as long as the anti-antibody has a function capable of recognizing the labeled antibody, that is, the anti-antibody has a function capable of capturing the labeled antibody that has developed along (permeated through) the development part 12 on the upstream side of the control portion 18. For example, any of IgG, IgA, IgM, IgE and IgD may be used, with IgG being preferably used from the viewpoint of ease of handling.

As such an anti-antibody, an anti-antibody that recognizes the constant region of the antibody (mouse antibody) included in the labeled antibody is usually used.

In addition, for the purpose of preventing aggregation between the labeled antibody and the human anti-mouse antibody (HAMA) from occurring, in general, a method in which a mouse antibody is added to the test sample (blood) in advance so that the mouse antibody is recognized by and adsorbed to the HAMA is known. However, the anti-antibody immobilized on the control portion 18 unintentionally recognizes the added mouse antibody, since the anti-antibody is used for recognizing the constant region of the antibody (mouse antibody) included in the labeled antibody. As a result, there are problems that the labeled antibody cannot be sufficiently recognized by the immobilized anti-antibody, and the reaction between the immobilized anti-antibody and the labeled antibody cannot be observed (visually recognized) in the control portion 18. Thus, an amount of the added mouse antibody is limited.

On the contrary, in a case where the labeled antibody in which the antigenic site recognized by the HAMA is defected or modified is used as the labeled antibody (the first labeled antibody and the second labeled antibody) which is labeled with the labeling substance, aggregation between the labeled antibody and the human anti-mouse antibody (HAMA) is prevented from occurring, so that the above-mentioned problems can be reliably prevented from occurring without adding a mouse antibody to the test sample.

In addition, immobilization of the anti-antibody on the control portion 18 can be carried out by using the same method as described in the immobilization of the specific antibody on the recognition portion 17.

The absorption portion 16 is in a strip shape in view of an overall shape thereof. And, the absorption portion 16 is disposed on the support substrate 11 so that an edge portion of the absorption portion 16 on an upstream side in a longitudinal direction thereof is in contact with (overlapping with) the development part 12. Thus, the absorption portion 16 absorbs (absorbs water in) the test sample which has developed along the development part 12.

Accordingly, the absorption portion 16 exerts a function of assisting the capillary phenomenon in the development part 12. For this reason, smoother development of the test sample from the upstream side to the downstream side in the development part 12 is made possible.

For the absorption portion 16, there is no particular limitation as long as the absorption portion 16 is liquid permeable. The absorption portion 16 can be formed from a porous material and a fibrous material in the same manner as the sample supply portion 14.

The housing case 20 has a waterproof property and includes a hollow portion 21 that corresponds to a shape of the test piece 10. The test piece 10 is accommodated in the hollow portion 21, to thereby facilitate a handling property of the test piece 10 (laboratory test kit 1).

The housing case 20 includes a test sample supply window 22 and a test result observation window 23. The housing case 20 is configured so that in a state where the test piece 10 is accommodated in the hollow portion 21, a portion (an upper surface) of the blood cell filtration portion 15 is exposed from the test sample supply window 22, and a portion of the development part 12 (a region where the recognition portion 17 and the control portion 18 are formed) is exposed from the test result observation window 23.

Accordingly, by dropping the test sample to the blood cell filtration portion 15 which is exposed from the test sample supply window 22, it is possible to supply the test sample to the sample supply portion 14 via the blood cell filtration portion 15. Then, by observing whether the labeled antibody is captured in the recognition portion 17 and the control portion 18 which are exposed from the test result observation window 23 and are provided on the development part 12, it is possible to diagnose the dengue virus infection.

For a constituent material for the housing case 20, there is no particular limitation as long as the material has the waterproof property and moderate strength. Examples thereof can include a resin material such as polystyrene, polycarbonate, polypropylene, and polyethylene terephthalate, and the like. Among these, one type can be used or two or more types can be used in combination.

Next, a method of using the above-described laboratory test kit 1, that is, a test method for confirming whether a subject is infected with the dengue virus will be described.

[1] First, whole blood, which is a test sample (specimen) containing the dengue virus and collected from a subject, is dropped to the blood cell filtration portion 15 which is exposed from the test sample supply window 22.

Accordingly, the test sample from which blood cell components have been removed, that is, plasma components are supplied to the sample supply portion 14 via the blood cell filtration portion 15. Furthermore, this test sample is transferred from the sample supply portion 14 to the reagent portion 13.

At this time, the labeled antibody (reagent) contained in the reagent portion 13 is brought into contact with the dengue virus antigen contained in the test sample. Due to such a contact, a complex composed of the labeled antibody and the dengue virus antigen is formed.

[2] Next, the test sample that has permeated through the reagent portion 13 is supplied to the development part 12 (first step).

Then, after being supplied so, the test sample develops along the development part 12 (second step). At this time, the complex also develops along the development part 12 and the dengue virus antigen is contained in this complex. Thus, the complex is captured by the specific antibody which is immobilized on the recognition portion 17 formed in the middle of the development part 12 and which specifically recognizes the dengue virus antigens.

Since the labeled antibody is contained in the complex together with the dengue virus antigen, it is possible to detect the dengue virus by observing (visually recognizing) the captured complex in the recognition portion 17.

[3] Next, the test sample that has permeated through the recognition portion 17 further reaches the control portion 18 which is positioned on the downstream side of the recognition portion 17 in the development part 12.

When the test sample reaches the control portion 18, the labeled antibody which is not involved in the formation of the complex is also contained in the test sample. Thus, such a labeled antibody is captured by the anti-antibody which is immobilized on the control portion 18 and recognizes the labeled antibody.

Therefore, by observing the captured labeled antibody in the control portion 18, it is possible to check that the detection result (determination result) that the subject is infected with the dengue virus which has been confirmed in the recognition portion 17 is correct.

The test sample that has permeated through the control portion 18 further develops toward the downstream side of the control portion 18 in the development part 12. And then, the test sample reaches the end portion of the development part 12, and finally is absorbed into the absorption portion 16.

By going through these steps using the above-described laboratory test kit 1, it is possible to know whether the dengue virus infection occurs or not in the subject.

In the present embodiment, addition of the labeled antibody to the test sample is achieved by a configuration in which the test piece 10 is configured to include the reagent portion 13, and the addition is achieved in a case where the test sample permeates through the reagent portion 13. However, the present invention is not limited to such a configuration. For example, prior to supply (dropping) of the test sample to the blood cell filtration portion 15, the labeled antibody may be directly added to the test sample in advance. In a case of such a configuration, the test piece 10 can be configured not to include the reagent portion 13.

Although a case of detecting the dengue virus which belongs to the family Flaviviridae has been described in the present embodiment, the present invention is not limited thereto. By appropriately selecting the specific antibody (first specific antibody and second specific antibody) and the labeled antibody (first labeled antibody and second labeled antibody) which are suitable for the respective viruses in order to obtain a detection kit according to the present invention, such a detection kit can also be applied to detection of other viruses. For example, the detection kit according to the present invention can also be applied to detection of other viruses which belong to the family Flaviviridae other than the dengue virus such as Japanese encephalitis virus, the family Orthomyxoviridae such as influenza virus, the family Paramyxoviridae such as measles virus, the family Retroviridae such as human immunodeficiency virus (HIV), the family Papillomaviridae such as papilloma virus and the family Reoviridae such as reovirus and rotavirus.

Up to here, although the laboratory test kit and the method for using the laboratory test kit of the present invention have been described based on the suitable embodiments, the present invention is not limited thereto.

For example, in the laboratory test kit of the present invention, each configuration can be replaced with any element which is capable of exerting an equivalent function, or an element having any configuration can be added thereto.

Furthermore, in the method for using the laboratory test kit, one or more steps can be added for any purpose.

### EXAMPLES

Next, specific examples of the present invention will be described.

1. Study of Window Size (Optimally Testable Period)

### Example 1A

1-1. Preparation of Specific Antibody and Labeled Antibody

(a) First Specific Antibody

As the first specific antibody derived from a mouse, an antibody in which both of an antigenic site recognized by a HAMA and a sugar chain binding site recognized by an antibody that recognizes a sugar chain are defected and which specifically recognizes the E protein included in the envelope that is possessed by the dengue virus was prepared.

(b) Second Specific Antibody

As the second specific antibody derived from a mouse, an antibody in which both an antigenic site recognized by a HAMA and a sugar chain binding site recognized by an antibody that recognizes a sugar chain are defected and which specifically recognizes NS1 which is produced by the dengue virus and is released to the outside of the virus was prepared.

(c) First Labeled Antibody

As the first labeled antibody derived from a mouse, a specific antibody labeled with colloidal gold in which both of an antigenic site recognized by a HAMA and a sugar chain binding site recognized by an antibody that recognizes a sugar chain are defected and which specifically recognizes the E protein included in the envelope that is possessed by the dengue virus was prepared.

(d) Second Labeled Antibody

As the second labeled antibody derived from a mouse, a specific antibody labeled with colloidal gold in which both of an antigenic site recognized by a HAMA and a sugar chain binding site recognized by an antibody that recognizes a sugar chain are defected and which specifically recognizes NS1 which is produced by the dengue virus and is released to the outside of the virus was prepared.

1-2. Manufacture of Laboratory Test Kit

1-2-1. Formation of Recognition Portion and Control Portion on Development Part

(a) Formation of Recognition Portion

Nitrocellulose (manufactured by Millipore Corporation) in a strip shape of 0.5 cm×3.0 cm was prepared as a development part. Further, at a position which is 1.5 cm from an end portion of the development part on an upstream side, 1.0 μL of a phosphate buffer solution in which each of the first specific antibody and the second specific antibody is contained in an amount of 5 mg/mL was added dropwise in a line shape having a width of about 0.1 cm in a direction orthogonal to a longitudinal direction of the development part. Thereafter, the phosphate buffer solution was dried at 60° C. for 4 hours to form a recognition portion. As a result, the first specific antibody and the second specific antibody were immobilized on the recognition portion in the middle of the development part.

(b) Formation of Control Portion

The control portion was formed at a position on a downstream side of the recognition portion in the middle of the development part in the same manner as in the formation of the recognition portion except the following points. That is, a phosphate buffer solution which contains an anti-antibody that recognizes the labeled antibody as described later in place of the first specific antibody and the second specific antibody was prepared. Further, such a phosphate buffer solution was added dropwise to a position which is 2.0 cm from the end portion of the development part on the upstream side.

1-2-2. Preparation of Reagent Portion

A porous carrier which is in a strip shape of 0.5 cm×1.0 cm and is composed of glass wool was prepared. Further, the porous carrier was impregnated in a phosphate buffer solution containing the first labeled antibody and the second labeled antibody. Then, the porous carrier was lyophilized to obtain a reagent portion in which the first labeled antibody and the second labeled antibody are held on the porous carrier.

1-2-3. Assembly of Laboratory Test Kit

First, a housing case of 2.7 cm×8.5 cm which has a hollow portion and is composed of polycarbonate, a support substrate of 0.5 cm×7.5 cm which is in a strip shape and is composed of white polystyrene, white vinyl, white polyester and clear polyester, a sample supply portion of 1.2 cm×1.9 cm which is in a strip shape and is composed of a glass filter (manufactured by Millipore Corporation), a blood cell filtration portion of 0.5 cm×1.0 cm which is in a strip shape and is formed of a porous carrier composed of glass wool, an absorption portion of 0.5 cm×3.0 cm which is in a strip shape and is formed of a porous carrier composed of filter paper and formed of a porous carrier composed of glass wool, a development part on which the recognition portion and the control portion are formed in the step 1-2-1, and the reagent portion obtained in the step 1-2-2 were respectively prepared.

Next, the sample supply portion, the reagent portion, the development part and the absorption portion were disposed on the support substrate in this order from the upstream side to the downstream side so that the edge portions thereof come into contact with each other. Then, the blood cell filtration portion was disposed on the sample supply portion, so that an immunochromatography test piece as shown in FIG. 2 was obtained.

Next, the obtained immunochromatography test piece was accommodated in the hollow portion of the housing case, so that a laboratory test kit as shown in FIG. 1 was assembled.

1-3. Diagnosis of Dengue Virus Infection

1-3-1. Target Cases

Subjects suspected of being infected with the dengue virus were targeted.

1-3-2. RT-PCR Method

For subjects who had been suspected of being infected with the dengue virus, the blood was collected as a specimen. Among these specimens, a total of 170 cases in which the dengue virus gene was detected using the RT-PCR method was used as a dengue virus-infected group (positive group).

For the specimens after 8 days from the onset, specimens collected from subjects which were determined to be positive by RT-PCR on or before 5 days were used.

1-3-3. Immunochromatography method (Example 1A)

For the subjects who had been suspected of being infected with the dengue virus, the blood (whole blood) was collected as a specimen on the same day (after 0 days), and the specimen was added dropwise on the blood cell filtration portion which is exposed from the test sample supply window included in the housing case of the laboratory test kit of Example 1A. Then, observation was made as to whether or not the labeled antibody is captured in the recognition portion and the control portion which are provided on the development part and exposed from the test result observation window.

As a result, capture of the labeled antibody in the recognition portion was observed regarding 9 cases (90%) out of the 10 cases which were used as a positive group in the step 1-3-2.

Thereafter, in these subjects, observation of capture of the labeled antibody in the recognition portion using the laboratory test kit of Example 1A was also made for after 1 day, after 2 days, after 3 days, after 5 days, after 8 days, after 10 days and after 12 days in the same manner as after 0 days.

As a result, the capture of the labeled antibody in the recognition portion was observed regarding 19 cases (95%) out of 20 cases for the after 1 day, 19 cases (95%) out of 20 cases for the after 2 days, 38 cases (95%) of 40 cases for the after 3 days, 38 cases (95%) out of 40 cases for the after 5 days, 18 cases (90%) out of 20 cases for the after 8 days, 9 cases (90%) of 10 cases for the after 10 days, and 8 cases (80%) out of 10 cases for the after 12 days.

### Comparative Example 1A

A laboratory test kit of Comparative Example 1A was manufactured in the same manner as in Example 1A except the following two points. As to the first point, a recognition portion on which the first specific antibody is immobilized is formed in the middle of the development part was prepared by omitting addition of the second specific antibody to the phosphate buffer solution at the time of forming the recognition portion in the step 1-2-1. (a). As to the second point, a reagent portion in which the first labeled antibody is held on the porous carrier was prepared by omitting addition of the second labeled antibody to the phosphate buffer solution at the time of preparing the reagent portion in the step 1-2-2. Then, using this laboratory test kit, the dengue virus infection was diagnosed.

### Comparative Example 2A

A laboratory test kit of Comparative Example 2A was manufactured in the same manner as in Example 1A except the following two points. As to the first point, a recognition portion on which the second specific antibody is immobilized is formed in the middle of the development part was prepared by omitting addition of the first specific antibody to the phosphate buffer solution at the time of forming the recognition portion in the step 1-2-1. (a). As to the second points, a reagent portion in which the second labeled antibody is held on the porous carrier was prepared by omitting addition of the first labeled antibody to the phosphate buffer solution at the time of preparing the reagent portion in the step 1-2-2. Then, using this laboratory test kit, the dengue virus infection was diagnosed.

The above results are shown in Table 1.

TABLE 1

| | Type of | tested time period (cases of being determined to be positive (rate | | | | | | | |
|---|---|---|---|---|---|---|---|---|---|
| | antigen | of being detected as being positive)) | | | | | | | |
| | recognized by | After | After | After | After | After | After | After | After |
| | antibody | 0 days | 1 day | 2 days | 3 days | 5 days | 8 days | 10 days | 12 days |
| Number of | | 10 | 20 | 20 | 40 | 40 | 20 | 10 | 10 |
| positive | | cases | cases | cases | cases | cases | cases | cases | cases |
| specimens | | | | | | | | | |
| Example 1A | NS1 + E | 9 | 19 | 19 | 38 | 38 | 18 | 9 | 8 |
| | protein | cases | cases | cases | cases | cases | cases | cases | cases |
| | | (90%) | (95%) | (95%) | (95%) | (95%) | (90%) | (90%) | (80%) |
| Comparative | E protein | 9 | 18 | 18 | 38 | 32 | 4 | 2 | 1 |
| Example 1A | | cases | cases | cases | cases | cases | cases | cases | case |
| | | (90%) | (90%) | (90%) | (95%) | (80%) | (40%) | (20%) | (10%) |
| Comparative | NS1 | 3 | 9 | 11 | 35 | 37 | 9 | 8 | 8 |
| Example 2A | | cases | cases | cases | cases | cases | cases | cases | cases |
| | | (30%) | (45%) | (55%) | (87.5%) | (92.5%) | (18%) | (80%) | (80%) |

As is apparent from Table 1, it has been shown that by using the laboratory test kit of Example 1A, the Dengue caused by the virus of the subjects can be diagnosed with excellent detection sensitivity at the window size ranging from the early stage to the long period such as after 0 to 10 days.

On the contrary, in the laboratory test kit of Comparative Example 1A, although the Dengue caused by the virus of the subjects can be diagnosed at the early stage such as after 0 to 5 days, the Dengue caused by the virus cannot be diagnosed with excellent detection sensitivity in the period later than after 5 days. In addition, in the laboratory test kit of Comparative Example 2A, although the Dengue caused by the virus of the subjects can be diagnosed during the period such as after 3 to 10 days, the Dengue caused by the virus cannot be diagnosed with excellent detection sensitivity at the early stage such as after 0 to 2 days.

2. Study of Antigen-Determining Site Recognized by HAMA

### Example 1B

2-1. Preparation of Specific Antibody and Labeled Antibody

(a) First Specific Antibody

As the first specific antibody derived from a mouse, an antibody in which an antigenic site recognized by a HAMA is defected and which specifically recognizes the E protein included in the envelope that is possessed by the dengue virus was prepared.

(b) Second Specific Antibody

As the second specific antibody derived from a mouse, an antibody in which an antigenic site recognized by a HAMA is defected and which specifically recognizes NS1 which is produced by the dengue virus and is released to the outside of the virus was prepared.

(c) First Labeled Antibody

As the first labeled antibody derived from a mouse, a specific antibody labeled with colloidal gold in which an antigenic site recognized by a HAMA is defected and which specifically recognizes the E protein included in the envelope that is possessed by the dengue virus was prepared.

(d) Second Labeled Antibody

As the second labeled antibody derived from a mouse, a specific antibody labeled with colloidal gold in which an antigenic site recognized by a HAMA is defected and which specifically recognizes NS1 which is produced by the dengue virus and is released to the outside of the virus was prepared.

2-2. Manufacture of Laboratory Test Kit

2-2-1. Formation of Recognition Portion and Control Portion on Development Part

(a) Formation of Recognition Portion

Nitrocellulose (manufactured by Millipore Corporation) in a strip shape of 0.5 cm×3.0 cm was prepared as a development part. Further, at a position which is 1.5 cm from an end portion of the development part on an upstream side, 1.0 μL of a phosphate buffer solution in which each of the first specific antibody and the second specific antibody is contained in an amount of 5 mg/mL was added dropwise in a line shape having a width of about 0.1 cm in a direction orthogonal to a longitudinal direction of the development part. Thereafter, the phosphate buffer solution was dried at 60° C. for 4 hours to form a recognition portion. As a result, the first specific antibody and the second specific antibody were immobilized on the recognition portion in the middle of the development part.

(b) Formation of Control Portion

The control portion was formed at a position on a downstream side of the recognition portion in the middle of the development part in the same manner as in the formation of the recognition portion except the following points. That is, a phosphate buffer solution which contains an anti-antibody that recognizes the labeled antibody as described later in place of the first specific antibody and the second specific antibody was prepared. Further, such a phosphate buffer solution was added dropwise to a position which is 2.0 cm from the end portion of the development part on the upstream side.

2-2-2. Preparation of Reagent Portion

A porous carrier which is in a strip shape of 0.5 cm×1.0 cm and is composed of glass wool was prepared. Further, the porous carrier was impregnated in a phosphate buffer solution containing the first labeled antibody and the second labeled antibody. Then, the porous carrier was lyophilized to obtain a reagent portion in which the first labeled antibody and the second labeled antibody are held on the porous carrier.

2-2-3. Assembly of Laboratory Test Kit

First, a housing case of 2.7 cm×8.5 cm which has a hollow portion and is composed of polycarbonate, a support substrate of 0.5 cm×7.5 cm which is in a strip shape and is composed of white polystyrene, white vinyl, white polyester or clear polyester, a sample supply portion of 1.2 cm×1.9 cm which is in a strip shape and is composed of a glass filter (manufactured by Millipore Corporation), a blood cell filtration portion of 0.5 cm×1.0 cm which is in a strip shape and is formed of a porous carrier composed of glass wool, an absorption portion of 0.5 cm×3.0 cm which is in a strip shape and is formed of a porous carrier composed of filter paper and formed of a porous carrier composed of glass wool, a development part on which the recognition portion and the control portion are formed in the step 1-2-1, and the reagent portion obtained in the step 1-2-2 were respectively prepared.

Next, the sample supply portion, the reagent portion, the development part and the absorption portion are disposed on the support substrate in this order from the upstream side to the downstream side so that the edge portions thereof come into contact with each other. Then, the blood cell filtration portion was disposed on the sample supply portion, so that an immunochromatography test piece as shown in FIG. 2 was obtained.

Next, the obtained immunochromatography test piece was accommodated in the hollow portion of the housing case, so that a laboratory test kit as shown in FIG. 1 was assembled.

2-3. Test as to Whether it is Determined to be False Positive

2-3-1. RT-PCR Method

For subjects who had been suspected of being infected with the dengue virus, the blood was collected as a specimen. Among these specimens, 30 cases were selected from the specimens in which the dengue virus gene was not detected using the RT-PCR method and which shows a false positivity by an immunochromatography method using wild-type mouse IgG. Furthermore, from the 30 cases, 20 cases shown to be highly positive by mouse IgG ELISA were used as a HAMA positive group.

2-3-2. Immunochromatography Method (Example 1B)

For the specimens (HAMA positive group) which were determined to be false positive due to having the HAMA activity, after dropping the specimen to the blood cell filtration portion exposed from the test sample supply window included in the housing case of the laboratory test kit of Example 1B, observation was made as to whether or not the labeled antibody is captured in the recognition portion and the control portion which are provided in the development part and exposed from the test result observation window.

As a result, capture of the labeled antibody in the recognition portion was observed regarding 3 cases (15%) out of 20 cases determined to be false positive (HAMA positive group) in the step 2-3-1.

### Comparative Example 1B

A laboratory test kit of Comparative Example 1B was manufactured in the same manner as in Example 1B except that the specific antibody and the labeled antibody as shown below were used in place of the specific antibody and the labeled antibody prepared in the step 2-1. Then, using this laboratory test kit, tests were carried out for the specimens (HAMA positive group) which were determined to be false positive due to having the HAMA activity.

(a) First Specific Antibody

As the first specific antibody derived from a mouse, an antibody which is normal from the viewpoint that an antigenic site recognized by a HAMA is not defected and which specifically recognizes the E protein included in the envelope that is possessed by the dengue virus was prepared.

(b) Second Specific Antibody

As the second specific antibody derived from a mouse, an antibody which is normal from the viewpoint that an antigenic site recognized by a HAMA is not defected and which specifically recognizes NS1 which is produced by the dengue virus and is released to the outside of the virus was prepared.

(c) First Labeled Antibody

As the first labeled antibody derived from mouse, a specific antibody labeled with colloidal gold which is normal from the viewpoint that an antigenic site recognized by a HAMA is not defected and which specifically recognizes the E protein included in the envelope that is possessed by the dengue virus was prepared.

(d) Second Labeled Antibody

As the second labeled antibody derived from a mouse, a specific antibody labeled with colloidal gold which is normal from the viewpoint that an antigenic site recognized by a HAMA is not defected and which specifically recognizes NS1 which is produced by the dengue virus and is released to the outside of the virus was prepared.

The above results are shown in Table 2.

TABLE 2

| | | Cases of being determined |
|---|---|---|
| | Antigenic site | to be false positive (rate |
| | recognized by | of being detected as being |
| | HAMA | false positive) |
| Example 1B | Defected | 3 cases (15%) |
| Comparative | Normal | 20 cases (100%) |
| Example 1B | | |

As is apparent from Table 2, by using the laboratory test kit of Example 1B, it was possible to carry out the test with excellent accuracy without making a determination as a false positivity for the subjects who had been determined to be false positive due to having the HAMA activity.

On the contrary, in the laboratory test kit of Comparative Example 1B, due to use of the specific antibody and the labeled antibody which are normal from the viewpoint that the antigenic site recognized by the HAMA is not defected, for the subjects who had been determined to be false positive due to having the HAMA activity, a result of being all determined to be false positive was shown.

3. Study of Sugar Chain Binding Site Recognized by Antibody that Recognizes Sugar Chain

### Example 1C

3-1. Preparation of Specific Antibody and Labeled Antibody

(a) First Specific Antibody

As the first specific antibody derived from a mouse, an antibody in which a sugar chain binding site recognized by an antibody that recognizes a sugar chain is defected and which specifically recognizes the E protein included in the envelope that is possessed by the dengue virus was prepared.

(b) Second Specific Antibody

As the second specific antibody derived from a mouse, an antibody in which a sugar chain binding site recognized by an antibody that recognizes a sugar chain is defected and which specifically recognizes NS1 which is produced by the dengue virus and released to the outside of the virus was prepared.

(b) First Labeled Antibody

As the first labeled antibody derived from a mouse, a specific antibody labeled with colloidal gold in which a sugar chain binding site recognized by an antibody that recognizes a sugar chain is defected and which specifically recognizes the E protein included in the envelope that is possessed by the dengue virus was prepared.

(b) Second Labeled Antibody

As the second labeled antibody derived from a mouse, a specific antibody labeled with colloidal gold in which a sugar chain binding site recognized by an antibody that recognizes a sugar chain is defected and which specifically recognizes NS1 which is produced by the dengue virus and released to the outside of the virus was prepared.

3-2. Manufacture of Laboratory Test Kit

3-2-1. Formation of Recognition Portion and Control Portion on Development Part

(a) Formation of Recognition Portion

Nitrocellulose (manufactured by Millipore Corporation) in a strip shape of 0.5 cm×3.0 cm was prepared as a development part. Further, at a position which is 1.5 cm from an end portion of the development part on an upstream side, 1.0 μL of a phosphate buffer solution in which each of the first specific antibody and the second specific antibody is contained in an amount of 5 mg/mL was added dropwise in a line shape having a width of about 0.1 cm in a direction orthogonal to a longitudinal direction of the development part. Thereafter, the phosphate buffer solution was dried at 60° C. for 4 hours to form a recognition portion. As a result, the first specific antibody and the second specific antibody were immobilized on the recognition portion in the middle of the development part.

(b) Formation of Control Portion

The control portion was formed at a position on a downstream side of the recognition portion in the middle of the development part in the same manner as in the formation of the recognition portion except the following points. That is, a phosphate buffer solution which contains an anti-antibody that recognizes the labeled antibody as described later in place of the first specific antibody and the second specific antibody was prepared. Further, such a phosphate buffer solution was added dropwise to a position which is 2.0 cm from the end portion of the development part on the upstream side.

3-2-2. Preparation of Reagent Portion

A porous carrier which is in a strip shape of 0.5 cm×1.0 cm and is composed of glass wool was prepared. Further, the porous carrier was impregnated in a phosphate buffer solution containing the first labeled antibody and the second labeled antibody. Then, the porous carrier was lyophilized to obtain a reagent portion in which the first labeled antibody and the second labeled antibody are held on the porous carrier.

3-2-3. Assembly of Laboratory Test Kit

First, a housing case of 2.7 cm×8.5 cm which has a hollow portion and is composed of polycarbonate, a support substrate of 0.5 cm×7.5 cm which is in a strip shape and is composed of white polystyrene, white vinyl, white polyester or clear polyester, a sample supply portion of 1.2 cm×1.9 cm which is in a strip shape and is composed of a glass filter (manufactured by Millipore Corporation), a blood cell filtration portion of 0.5 cm×1.0 cm which is in a strip shape and is formed of a porous carrier composed of glass wool, an absorption portion of 0.5 cm×3.0 cm which is in a strip shape and is formed of a porous carrier composed of filter paper and formed of a porous carrier composed of glass wool, a development part on which the recognition portion and the control portion are formed in the step 1-2-1, and the reagent portion obtained in the step 1-2-2 were respectively prepared.

Next, the sample supply portion, the reagent portion, the development part and the absorption portion were disposed on the support substrate in this order from the upstream side to the downstream side so that the edge portions thereof come into contact with each other. Then, the blood cell filtration portion was disposed on the sample supply portion, so that an immunochromatography test piece as shown in FIG. 2 was obtained.

Next, the obtained immunochromatography test piece was accommodated in the hollow portion of the housing case, so that a laboratory test kit as shown in FIG. 1 was assembled.

3-3. Test as to Whether it is Determined to be False Positive

3-3-1. RT-PCR Method

For subjects who had been suspected of being infected with the dengue virus, the blood was collected as a specimen. Among these specimens, 30 cases in which the dengue virus gene was not detected using the RT-PCR method was set as a dengue virus negative group. Furthermore, 20 cases which were ELISA negative for a recombinant E protein produced by E. coli were used as sugar chain-binding specimens.

3-3-2. Immunochromatography Method (Example 1C)

Using the specimen (sugar chain-binding specimen) which was determined to be false positive due to having the antibody that recognizes the sugar chain included in the sugar chain binding site, after dropping the specimen to the blood cell filtration portion exposed from the test sample supply window included in the housing case of the laboratory test kit of Example 1C, observation was made as to whether or not the labeled antibody is captured in the recognition portion and the control portion which are provided in the development part and exposed from the test result observation window.

As a result, capture of the labeled antibody in the recognition portion was observed regarding 2 cases (10%) out of 20 cases (sugar chain-binding specimens) which had been determined to be false positive in the step 3-3-1.

### Comparative Example 1C

The laboratory test kit of Comparative Example 1C was manufactured in the same manner as in Example 1C except that the specific antibody and the labeled antibody as shown below were used in place of the specific antibody and the labeled antibody prepared in the step 3-1. Then, using this laboratory test kit, tests were carried out for the specimens (sugar chain-binding specimens) which had been determined to be false positive due to having the antibody that recognizes the sugar chain included in the sugar chain binding site.

First Specific Antibody

As the first specific antibody derived from a mouse, an antibody which is normal from the viewpoint that a sugar chain binding site recognized by an antibody that recognizes a sugar chain is not defected and which specifically recognizes the E protein included in the envelope that is possessed by the dengue virus was prepared.

(b) Second Specific Antibody

As the second specific antibody derived from a mouse, an antibody which is normal from the viewpoint that a sugar chain binding site recognized by an antibody that recognizes a sugar chain is not defected and which specifically recognizes NS1 which is produced by the dengue virus and released to the outside of the virus was prepared.

First Labeled Antibody

As the first labeled antibody derived from a mouse, a specific antibody labeled with colloidal gold which is normal from the viewpoint that a sugar chain binding site recognized by an antibody that recognizes a sugar chain is not defected and which specifically recognizes the E protein included in the envelope that is possessed by the dengue virus was prepared.

Second Labeled Antibody

As the second labeled antibody derived from a mouse, a specific antibody labeled with colloidal gold which is normal from the viewpoint that a sugar chain binding site recognized by an antibody that recognizes a sugar chain is not defected and which specifically recognizes NS1 which is produced by the dengue virus and released to the outside of the virus was prepared. The above results are shown in Table 3.

TABLE 3

| | Sugar binding site | Cases of being determined |
|---|---|---|
| | recognized by antibody | to be false positive (rate |
| | that recognizes | of being detected as being |
| | sugar chain | false positive) |
| Example 1C | Defected | 2 cases (10%) |
| Comparative | Normal | 20 cases (100%) |
| Example 1C | | |

As is apparent from Table 3, by using the laboratory test kit of Example 1C, it was possible to carry out the test with excellent accuracy without making a determination as a false positivity for the subjects who had been determined to be false positive due to having the antibody that recognizes the sugar chain included in the sugar chain binding site.

On the contrary, in the laboratory test kit of Comparative Example 1C, due to use of the specific antibody and the labeled antibody which are normal from the viewpoint that the sugar chain binding site recognized by the antibody that recognizes the sugar chain is not defected, for the subjects who had been determined to be false positive due to having the antibody that recognizes the sugar chain included in the sugar chain binding site, a result of being all determined to be false positive was shown.

In each of the above examples, the studies were made for a case where, the antibodies in which at least one of the antigenic site recognized by the HAMA or the sugar chain binding site recognized by the antibody that recognizes the sugar chain is defected were used as the specific antibody and the labeled antibody. However, for a case where antibodies in which at least one of the antigenic site recognized by the HAMA or the sugar chain binding site recognized by the antibody that recognizes the sugar chain is modified were used, studies were also made, and even for this case, the same results as in the case of defect were obtained.

### INDUSTRIAL APPLICABILITY

The laboratory test kit of the present invention is used for diagnosing the infection caused by a virus, and has a development part for developing a test sample that contains the virus, a recognition portion provided in the middle of the development part, and a reagent portion disposed so as to be in contact with an edge portion of the development part on an upstream side in a case where a development direction in which the test sample is developed along the development part is taken as a reference. On the recognition portion, a first specific antibody and a second specific antibody which are derived from a mouse and specifically recognize a first antigen-determining site and a second antigen-determining site of the virus, respectively, are immobilized. In the reagent portion, a first labeled antibody and a second labeled antibody which are derived from a mouse, and are labeled with a labeling substance and have a function of specifically recognizing the first antigen-determining site and the second antigen-determining site of the virus, respectively, are contained, and, in each of the first labeled antibody and the second labeled antibody, at least one of an antigenic site recognized by a HAMA or a sugar chain binding site recognized by an antibody that recognizes a sugar chain is defected or modified. Therefore, Dengue caused by the virus can be diagnosed at early days after onset with excellent detection sensitivity. In this way, early diagnosis of Dengue can be carried out with excellent accuracy, and a therapeutic policy can be established at an early stage. Therefore, in a case where such a test for diagnosing Dengue is applied to the diagnosis of the dengue virus infection, it is possible to reduce onset of dengue hemorrhagic fever/dengue shock syndrome (DHF/DSS), and furthermore, a fatality rate caused by the DHF/DSS onset. Therefore, the laboratory test kit and the method for using the laboratory test kit of the present invention have industrial applicability.

### REFERENCE SIGNS LIST

1: Laboratory test kit
10: Immunochromatography test piece (test piece)
11: Support substrate
12: Development part
13: Reagent portion
14: Sample supply portion
15: Blood cell filtration portion
16: Absorption portion
17: Recognition portion
18: Control portion
20: Housing case
21: Hollow portion
22: Test sample supply window
23: Test result observation window

1. A laboratory test kit used for diagnosing infection caused by a virus, comprising a development part for developing thereon a test sample containing the virus, a recognition portion provided in the middle of the development part, and a reagent portion disposed in contact with an edge portion of an upstream side of the development part when a developing direction in which the test sample is developed on the development part is taken as a reference, the recognition portion on which a first specific antibody and a second specific antibody both derived from a mouse are to be immobilized for specifically recognizing a first antigen-determining site and a second antigen-determining site both possessed by the virus, the reagent portion includes a first labeled antibody and a second labeled antibody both derived from a mouse, which have a function of specifically recognizing the first antigen-determining site and the second antigen-determining site both possessed by the virus and are labeled with a labeling substance, and in each of the first labeled antibody and the second labeled antibody, at least one of an antigen site which is recognized by a human anti-mouse antibody or a sugar chain binding site recognized by an antibody which recognizes a sugar chain is defected or modified.

2. The laboratory test kit as claimed in claim 1, wherein in each of the first specific antibody and the second specific antibody, at least one of an antigen site which is recognized by the human anti-mouse antibody or a sugar chain binding site recognized by the antibody which recognizes the sugar chain is defected or modified.

3. The laboratory test kit as claimed in claim 1, wherein the virus is dengue virus.

4. The laboratory test kit as claimed in claim 3, wherein each of the first specific antibody and the first labeled antibody specifically recognizes a part of an internal antigen possessed by the dengue virus.

5. The laboratory test kit as claimed in claim 4, wherein the internal antigen is an E-protein contained in an envelope.

6. The laboratory test kit as claimed in claim 3, wherein each of the second specific antibody and the second labeled antibody specifically recognizes a part of an external antigen possessed by the dengue virus.

7. The laboratory test kit as claimed in claim 6, wherein the external antigen is a NS1 produced by the dengue virus at the outside of the virus.

8. The laboratory test kit as claimed in claim 1, further comprises a sample supply portion disposed so as to be in contact with the edge portion of the upstream side of the reagent portion, and a blood-corpuscles filtering portion disposed so as to be laminated on the sample supply portion, wherein the test sample is whole blood, wherein the laboratory test kit is configured so that by supplying the test sample into the sample supply portion through the blood-corpuscles filtering portion, the blood-corpuscles components contained in the test sample are eliminated.

9. A method for diagnosing virus infection using the laboratory test kit defined in claim 1, comprising:
a first step for supplying the test sample onto the developing layer; and a second step for confirming whether or not the virus is captured by the recognition portion provided in the middle of the development part when the test sample is developed onto the development part.
